(19) United States
(12) Patent Application Publication
(10) Pub. No.: US 2020/0078509 A1
(43) Pub. Date: Mar. 12, 2020

(54) MICRO-DISPENSER BASED EAR TREATMENT

(71) Applicant: Xinova, LLC, Seattle, WA (US)

(72) Inventors: Mateusz Bryning, San Jose, CA (US); Remy Cromer, Saratoga, CA (US)

(73) Assignee: Xinova, LLC, Seattle, WA (US)

(21) Appl. No.: 16/128,582

(22) Filed: Sep. 12, 2018

(51) Int. Cl. A61M 3/02; A61M 3/00; A61B 1/227; A61K 9/00; A61K 9/08

(52) U.S. Cl. CPC A61M 3/0279; A61M 2205/502; A61B 1/227; A61M 3/0275; A61K 9/0046; A61K 9/08; A61M 2210/0662; A61M 2205/3379; A61M 2205/3331; A61M 2205/3368; A61M 2205/3303; A61M 2205/3653; A61M 2205/52; A61M 2205/581; A61M 3/005

(57) ABSTRACT

Technologies are generally described for treatment of ear canal through a micro-dispenser-based treatment system. In some examples, ear canal irrigating fluids may be dispensed in a controlled volume and pressure through one or more micro-dispensers to dissolve and remove ear wax in a gentle and safe manner without harming the eardrum. Through positioning of the micro-dispensers or nozzles of the micro-dispensers, the fluid may be aimed directly at specific areas within the ear canal with a high degree of precision. The micro-dispenser(s) and an optional imaging device such as a camera may be integrated with an otoscope or a cannula for manual manipulation as well as for aiming the fluid jet with visual assessment of cleaning effectiveness

### BACKGROUND

Unless otherwise indicated herein, the materials described in this section are not prior art to the claims in this application and are not admitted to be prior art by inclusion in this section.

Ear wax, also known as cerumen, is a yellowish waxy substance secreted in the ear canal of humans and other mammals to protect the skin of the ear canal and provide some protection against bacteria, fungi, insects, and water. However, excessive ear wax may impede the passage of sound in the ear canal, causing conductive hearing loss. Excess or compacted ear wax can also press against the eardrum or block the outside ear canal or hearing aids, potentially causing hearing loss. With the increase usage of earphones or earbuds, increased hearing loss may be expected.

Ear wax removal may be difficult through mechanical extraction, which may also pose a risk of hurting the eardrums. Commonly used cotton swabs, for example, may push most of the ear wax farther into the ear canal and remove only a small portion of the top layer of wax. Furthermore, cotton swabs or similar item, if used carelessly, may perforate the eardrum. Medical professionals recommend ear irrigation by syringing with solutions containing softeners. This may be accomplished with a spray type ear washer in a medical setting or with a bulb syringe at home. Both approaches lack proper control of fluid pressure, however, and may result in damage to the eardrums.

### SUMMARY

The present disclosure generally describes devices and techniques to treat the ear canal through a micro-dispenser based system.

According to some examples, a method for ear canal treatment through a micro-dispenser-based system is described. The method may include determining one or more of a volume and a pressure for fluid to be directed to an ear canal; determining a region of the ear canal to be targeted; and directing the fluid to the region of the ear canal through one or more micro-dispensers based on the one or more of the volume and the pressure.

According to other examples, a micro-dispenser based ear canal treatment system is described. The treatment system may include a delivery module comprising a supply sub-system and a dispensing sub-system, and a controller. The delivery module may direct a fluid to a selected region of an ear canal through one or more micro-dispensers. The controller may be configured to determine one or more of a volume and a pressure for the fluid to be directed to the ear canal; and determine the region of the ear canal to be targeted.

According to further examples, an otoscope capable of providing ear canal treatment is described. The otoscope may include one or more reservoirs configured to store one or more fluids and one or more micro-dispensers. The micro-dispensers may be configured to receive the one or more fluids from the one or more reservoirs; and direct the one or more fluids to a selected region of an ear canal at a predetermined volume and pressure through one or more nozzles, where the one or more nozzles are positioned at a tip of the otoscope.

According to some examples, a cannula capable of providing ear canal treatment is described. The cannula may include one or more micro-dispensers coupled to one or more reservoirs configured to store one or more fluids. The one or more micro-dispensers may be configured to receive the one or more fluids from the one or more reservoirs; and direct the one or more fluids to a selected region of an ear canal at a predetermined volume and pressure through one or more nozzles, where the one or more nozzles are positioned at a tip of the cannula.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates an example ear anatomy with a micro-dispenser-based treatment system;

FIGS. 2A-2B illustrate an example micro-dispenser-based ear canal treatment system in various configurations;

FIG. 3 illustrates an example micro-dispenser-based ear canal treatment system in an otoscope configuration;

FIGS. 4A-4B illustrate different configurations of major components in an example micro-dispenser based ear canal treatment system;

FIG. 5 is a block diagram illustrating major components in an example remotely controlled micro-dispenser based ear canal treatment system;

FIG. 6 illustrates a computing device, which may be used to control a micro-dispenser-based ear canal treatment system;

FIG. 7 is a flow diagram illustrating an example method to treat the ear canal through a micro-dispenser-based treatment system that may be performed by a computing device such as the computing device in FIG. 6; and

FIG. 8 illustrates a block diagram of an example computer program product, all arranged in accordance with at least some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to methods, apparatus, systems, devices, and/or computer program products related to treatment of ear canal through a micro-dispenser based system.

Briefly stated, technologies are generally described for treatment of ear canal through a micro-dispenser-based treatment system. In some examples, ear canal irrigating fluids may be dispensed in a controlled volume and pressure through one or more micro-dispensers to dissolve and remove ear wax in a gentle and safe manner without harming the eardrum. Through positioning of the micro-dispensers or nozzles of the micro-dispensers, the fluid may be aimed directly at specific areas within the ear canal with a high degree of precision. The micro-dispenser(s) and an optional imaging device such as a camera may be integrated with an otoscope or a cannula for manual manipulation as well as for aiming the fluid jet with visual assessment of cleaning effectiveness.

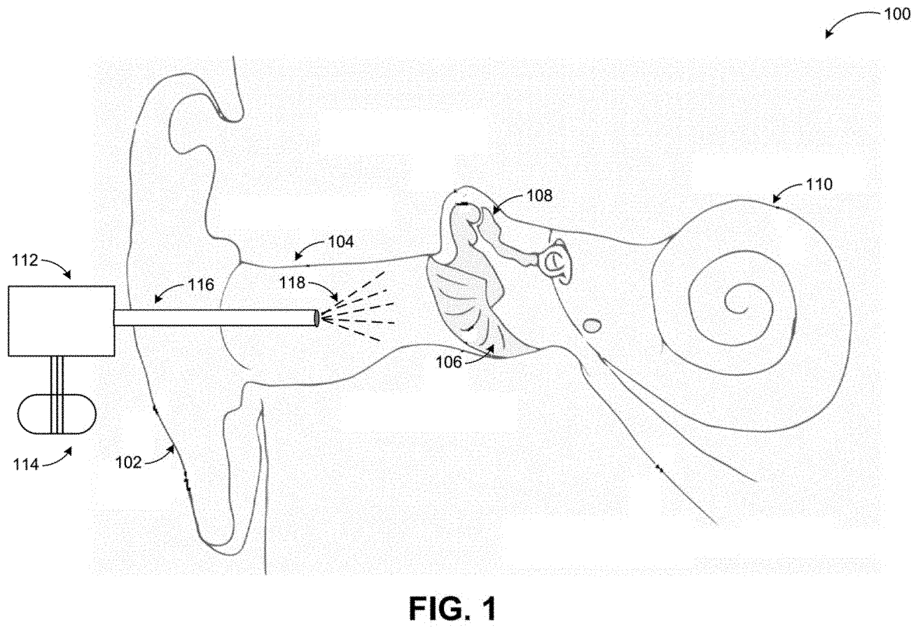

FIG. 1 illustrates an example ear anatomy with a micro-dispenser-based treatment system, arranged in accordance with at least some embodiments described herein.

Diagram 100 shows a simplified ear anatomy that includes the outer ear 102, ear canal 104, middle ear comprising tympanic membrane (ear drum) 106 and ossicles 108 (malleus, incus, and stapes), and cochlea (inner ear) 110. Other parts of the ear such as semi-circular canals, vestibular nerve, eustachian tube, portions of the outer ear, etc. are not shown in detail for sake of simplicity.

Ear wax is typically produced in the outer third of the cartilaginous portion of the ear canal. It is a mixture of shed skin cells, hair, and viscous secretions from sebaceous glands and less-viscous ones from modified apocrine sweat glands. Major components of ear wax may include shed layers of skin and an average of 60% of the earwax consisting of keratin, 12-20% saturated and unsaturated long-chain fatty acids, alcohols, squalene and 6-9% cholesterol. Excess or compacted cerumen may press against the ear drum or block the outside ear canal or hearing aids, potentially causing hearing loss.

While a number of methods of ear wax removal are effective, their comparative merits have not been determined. A number of softeners have been shown as effective with the most common method of cerumen removal being syringing with warm water. Curette methods are also used by otolaryngologists when the ear canal is partially occluded and the material is not adhering to the skin of the ear canal. Cotton swabs, on the other hand, may push most of the ear wax farther into the ear canal and remove only a small portion of the top layer of wax that happens to adhere to the fibers of the swab, and are, therefore not recommended for use.

According to some embodiments, a micro-dispenser based device may be used to deliver fluids to the ear canal to aid removal of ear wax and/or treat other conditions. For example, compounds (e.g., drugs or drug combinations) may be delivered through the ear canal to treat conditions of the inner ear or other parts of the body, damages on the ear canal may be treated by delivery of sealants or similar compounds, ear infections or infestations may be treated, among other things. For example, various antibiotics (in liquid form) may be delivered to the ear canal, sometimes in combination with other agents such as hydrocortisone, triamcinolone acetonide, alcohol, or other steroids, to treat bacterial infections. Similarly, antifungal drugs may also be delivered. In other example implementations, drying agents may be delivered (e.g., alcohol solutions, acetic acid, etc.) to dry the ear canal and prevent microbial growth in a dark and humid environment. Other compounds that may be delivered by a system according to embodiments may include natural antibiotics (e.g., garlic based solutions, olive oil, or glycerin-benzocaine combinations), as well as, pain reducers to treat pain from ear infections. In yet other examples, cyanoacrylate glue or similar compounds may be delivered to treat perforations of the ear drum.

In a basic configuration, the micro-dispenser based device may include a delivery module and a supply sub-system. The supply sub-system may comprise a repository 114 or multiple repositories. The delivery module may include a dispensing sub-system 112 (e.g., one or more micro-dispensers) and a nozzle 116 configured to direct the fluid(s) in a predefined or adjustable spray pattern 118.

The fluid(s) may be dispensed according to one or more dispensation parameters, which may include a dispensed volume, a pressure, a direction, a fluid temperature, a dispensation pattern, a fluid composition, or a nozzle selection. The dispensation parameters may be determined manually (e.g., based on visual observation) or automatically based on factors such as a size of a build-up of an ear wax in the ear canal, a composition of the ear wax, a shape of the ear canal, a depth of the ear canal, and a thickness of the ear drum. In some example implementations, a mechanical element (e.g., a vibration element) such as oscillation of spray paths, rotating cams, sonic or ultrasonic beams, etc. may be integrated with the micro-dispenser based device to help removal of ear wax build-up. Examples of mechanical elements may include piezoelectric actuators, motors (e.g., servo motors), MEMS actuators, shape memory alloys (e.g., TiNi, aka nitinol), etc. Any of these elements may also be used to direct the fluid dispensing.

In other examples, a visual observation element such as a camera may also be integrated with the device for initial observations (to determine the dispensation parameters), as well as, for follow-up observations to detect effectiveness of treatment. Observation and analysis of ear wax may provide other medical advantages as well. For example, evidence of a strong fluorescence signal form cerumen in the external auditory canal at multiple excitation wavelengths has been observed. Thus, ear wax may be identified by autofluorescence. Additionally, other problems of the inner ear may be detected with a fluorescence otoscope, and treated with the microdispenser based device (that is, suitable compounds and delivery parameters may be selected based on the detection).

Furthermore, diagnostic biomarkers such as genetic material, lipids, proteins, chemical elements, internal and external metabolites (e.g., hormones, volatile organic compounds, amino acids, xenobiotics etc.) may reach ear wax from the blood circulation. Thus, ear wax is able to reflect not only physiology and pathophysiology of the body, but can also detect recent and long-term exposure to environmental pollutants without a need for invasive blood tests or collection of other diagnostic biological fluids. In this context, a device according to embodiments may be used to help harvest ear wax for diagnostic purposes (e.g., with the help of the above-discussed mechanical elements).

In another embodiment, the microdispenser based device may be used to conduct diagnostics in-situ. The microdispenser(s) may apply a substance into the ear that selectively stains bacteria, lesions, cholesterol, etc., which may then be detected in-situ with a fluorescence otoscope. The identification of a specific condition may be followed by appropriate treatment (e.g., antibiotic spray in case of bacterial infection). In other cases, such as visualization of cholesterol. for example, the staining/fluorescence imaging may provide diagnostic information about a medical condition without applying a follow-on treatment in-situ.

In some examples, computing capability may be provided to the microdispenser based device through an integrated processor or through near-field communications, and computer vision may be used to identify ear wax by its color and/or shape and to highlight on the screen. Thus, ear wax identification may be accomplished, not only through staining, but also through image processing. In other examples, the device may be communicatively linked to a smartphone or other external display, where the images or video from the otoscope may be displayed, optionally with problem areas highlighted, such that a user can actuate the delivery elements of the device. This feature may enable the user to administer the treatment singlehandedly without a second person. Moreover, a device with discussed features may be easier to utilize in a home environment. For example, a parent may administer treatment to a child, while both can monitor progress via a smartphone display.

A shape of spray distribution for the fluid(s) may be adjusted based on one or more of a location of a build-up of an ear wax in the ear canal, a size of the build-up of the ear wax in the ear canal, a composition of the ear wax, a shape of the ear canal, a depth of the ear canal, and a thickness of the ear drum. The shape of spray distribution for the fluid may be adjusted by activating one or more nozzles associated with the one or more micro-dispensers. The fluid(s) may be directed to a region of the ear canal through the one or more micro-dispensers. The device in its entirety or portions of the device (e.g., the nozzles or the dispensing unit) may be integrated with an otoscope or a cannula.

The dispensed fluid(s) may include a softening agent such as a plant-based oil, a synthetic oil, urea hydrogen peroxide, glycerin, sodium bicarbonate solution in water, sodium bicarbonate and glycerin solution (B.P.C.), peanut oil turpentine and dichlorobenzene, triethanolamine polypeptides and oleate-condensate, warm water, saline solution, water and vinegar solution, or various combinations thereof. In some examples, a softening agent may be delivered first, followed by a rinsing agent (e.g., warm water), optionally combined with mechanical action to loosen and remove ear wax.

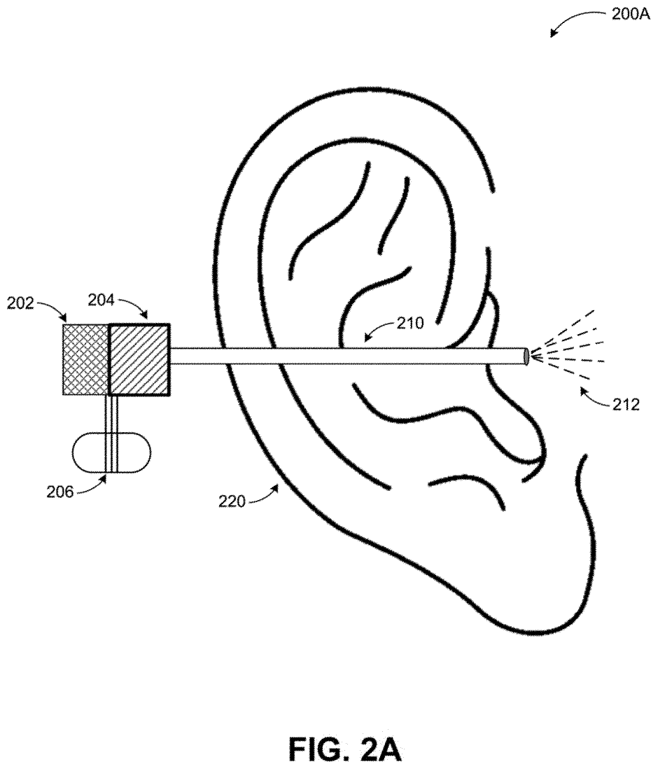

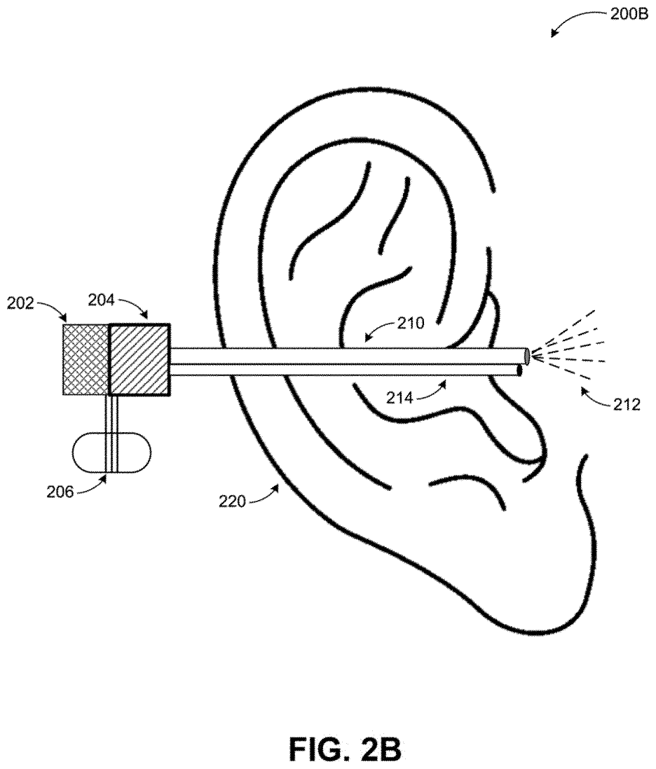

FIGS. 2A-2B illustrate an example micro-dispenser-based ear canal treatment system in various configurations, arranged in accordance with at least some embodiments described herein.

Diagram 200A of FIG. 2 shows an example configuration of a micro-dispenser-based ear canal treatment system that includes a fluid repository 206, a dispensing unit 204, a controller 202, and one or more nozzles 210 directing one or more fluids with a selected spray pattern 212 into the ear canal of ear 220.

As discussed above, the fluid(s) may include a number of softening agents, rinsing agents, drugs, or similar compounds and combinations thereof. Thus, repository 206 may include a single repository or multiple repositories for different fluids. The repository 206 may be for one-time-use or refillable. Repository 206 may also be replaceable. For example, different cartridges filled with different fluids may be used and replaced.

Dispensing unit 204 may include one or more micro-dispensers. The micro-dispensers may be arranged in an array format and configured to direct the fluids concurrently or sequentially according to selected dispensation parameters. The micro-dispensers may include a Joule-heating dispenser, for example. A Joule-heating dispenser may contain a fraction of the liquid sample in a conical reservoir, according to at least some embodiments. The Joule-heating dispenser, which may apply energy to a fluid reservoir in the form of heat, which may cause parts of the fluid in contact with the reservoir to evaporate, forming one or more vapor bubbles. The formation and collapse of the vapor bubbles may create a unidirectional dispensing action. In such embodiments, the volume and/or pressure of the fluid dispensed may be dictated by the amount of energy applied to the reservoir of the micro-dispenser or by the amount of time the energy is applied to the reservoir. In other embodiments, the micro-dispensers may include a piezo-electric dispenser. A piezo-electric dispenser may contain a fraction of the fluid in a reservoir. An electrical potential may be applied to the dispenser resulting in a mechanical force proportional to the electrical potential to be applied to the fluid in the reservoir. As a result, the fluid may be extruded or dispensed through an orifice in the reservoir.

The nozzles 210 may include one or more nozzles coupled to the micro-dispensers. In some examples, a direction of the nozzles may be adjustable to achieve a desired spray pattern. The pressure, volume, and type (in case of multiple fluids) of the fluid(s) to be directed may also be controlled by controlling the nozzles (e.g., their direction, opening, selection, etc.). As shown in diagram 200A, the nozzles 210 or the entire device may be incorporated into a cannula which may be inserted into the ear canal.

Controller 202 may include a processor to determine the dispensation parameters based on automatic observation (e.g., using a camera or other detection methods), direct user input through buttons or other input mechanisms, or remote input transmitted to the controller 202 from a remote device through wired or wireless means. In the scenario of a remote device connection, the controller 202 may also include a communication module for wired or wireless communications. Controller 202 may also observe fluid levels, effectiveness of the fluid dispensation (e.g., through a camera), and provide feedback to a user (e.g., a medical professional) through wired or wireless communication or a display device coupled to the controller (not shown).

Diagram 200B of FIG. 2B shows another configuration of a micro-dispenser based ear canal treatment system, where in addition to the example configuration of FIG. 2A, an observation device 214 (e.g., a camera) is incorporated along with the nozzles 210 of the device. The camera may be positioned such that observations may be made through the tip of the cannula in the example configuration allowing a user to determine the dispensation parameters, observe an effectiveness of the treatment, and adjust the dispensation parameters based on the observations. Similarly, controller 202 may perform some or all of the observations and determine or adjust the dispensation parameters automatically. In some examples, a combination of automatic and manual determination and adjustment may be performed. The device may also include a power source (not shown) such as a battery, a direct current (DC) source, or an alternative current (AC) source.

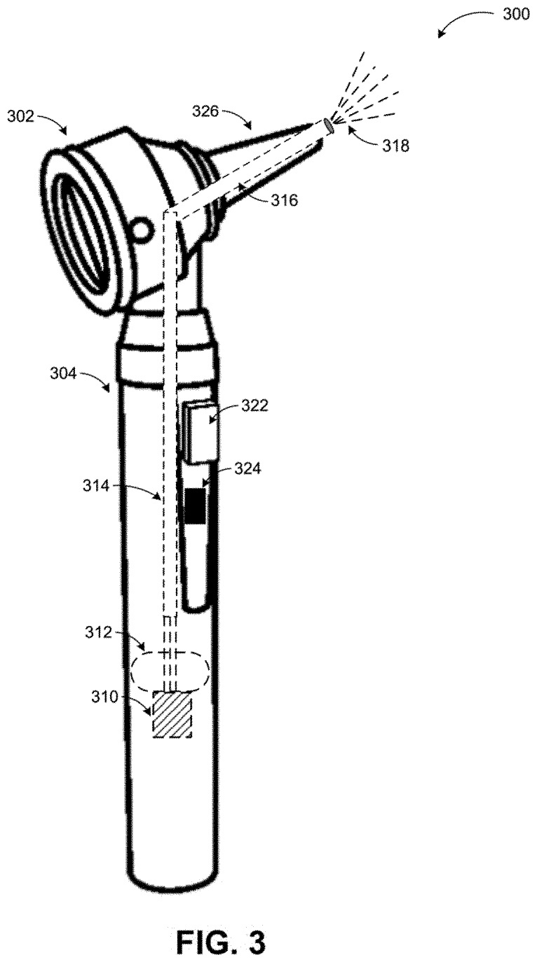

FIG. 3 illustrates an example micro-dispenser-based ear canal treatment system in an otoscope configuration, arranged in accordance with at least some embodiments described herein.

Diagram 300 shows an example otoscope configuration, where the micro-dispenser-based ear canal treatment is integrated with an otoscope. A dispensing unit 310 integrated with a controller, a fluid repository 312, and a delivery tube 314 connecting the dispensing unit 310 to one or more nozzles 316 may be positioned in a tail section 304 of the otoscope, while the one or more nozzles 316 may be placed inside a head section 302 of the otoscope such that the nozzles spray the fluid(s) through the tip of a speculum 326 of the otoscope with a spray pattern 318. One or more buttons, sliders, or similar input controls 322 may also be placed on the tail section 304 to allow a user to provide input to the controller. In some examples, a display 324 to provide visual feedback on progress and dispensation parameters (and/or a speaker for audio feedback) may also be integrated to the tail section 304 of the otoscope.

The controller may allow programming of the dispensation parameters (related to dispensation of the fluid) such as dispensed volume, pressure, direction, fluid temperature, controlled dispensation at intervals (e.g., dynamic control of irrigation pulses, pulsed dispensation), fluid composition (e.g., concentration of an active ingredient or combination of compounds/agents), nozzle selection (e.g., short dispensation time using all nozzles, longer dispensation time for fewer nozzles, use of different nozzles for different fluids. etc.).

The dispensed fluids may include drugs or compounds to treat various conditions of the ear or other body parts (by infusion through the ear drum or the ear canal) and softening and/or rinsing agents for removal of ear wax. For example, softening agents may include a plant-based oil, a synthetic oil, urea hydrogen peroxide, glycerin, sodium bicarbonate solution in water, sodium bicarbonate and glycerin solution (B.P.C.), peanut oil turpentine and dichlorobenzene, triethanolamine polypeptides and oleate-condensate, warm water, saline solution, water and vinegar solution, or various combinations thereof. Rinsing agents may include a subset of the softening agent such as warm water.

In addition to the magnifying glass in the head section 302 of the otoscope for visual inspection, a camera may also be incorporated to the tip of the otoscope's speculum 326 for manual or automatic observation. The otoscope may also be equipped with a power source (not shown) such as a battery, a direct current (DC) source, or an alternative current (AC) source.

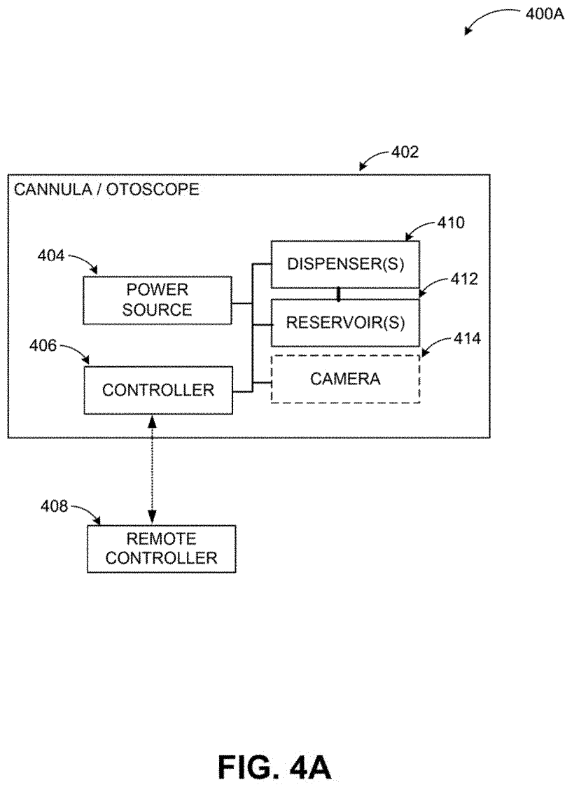

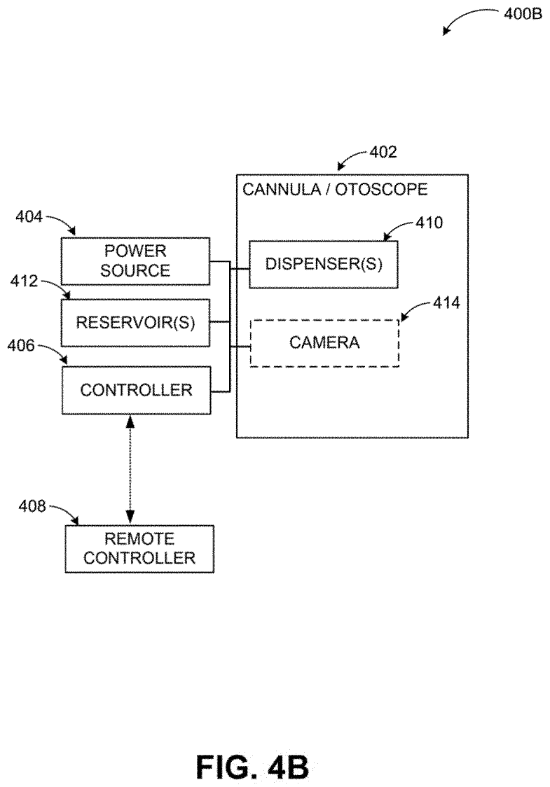

FIGS. 4A-4B illustrate different configurations of major components in an example micro-dispenser based ear canal treatment system, arranged in accordance with at least some embodiments described herein.

Various components of a micro-dispenser based ear canal treatment system may be combined in practical implementations. As shown in diagram 400A, a cannula or otoscope 402 may include micro-dispenser(s) 410, repository (reservoir(s) 412), controller 406, power source 404, and an optional camera 414. The controller 406 may communicate with a remote controller 408 to receive instructions (e.g., dispensation parameters), provide observation results (e.g., a video stream), and/or provide feedback (e.g., observations of effectiveness of the treatment, fluid levels, power level, etc.).

Various combinations of repository, micro-dispenser, and nozzles may be implemented. For example, a single repository may feed an array of micro-dispensers, each micro-dispenser may be coupled to its dedicated repository, each micro-dispenser may have its own nozzle, or each micro-dispenser may be coupled to multiple nozzles. Dispensation parameters such as volume, pressure, timing, spray pattern, etc. may be set or adjusted by controlling the repository, micro-dispenser(s), nozzle(s), and combinations thereof.

Diagram 400B of FIG. 4 shows another example configuration, where cannula or otoscope 402 includes the micro-dispenser(s) 410 and the optional camera 414 with the power source 404, reservoir(s) 412, and controller 406 being embodied outside the body of the cannula or otoscope 402. The external components may be coupled to the internal components through wired or wireless means with the exception of the reservoir(s) 412 providing the fluid(s) to the micro-dispenser(s) 410.

In some embodiments, the micro-dispenser based ear canal treatment system may include aspects that may allow a medical professional to decide on the dispensation parameters and then program them into the system. For example, a doctor may view an image of the ear canal captured through an image sensor on the device and then program the dispensation parameters into the system based on the image. Selection of some dispensation parameters may be manual, automated, or semi-automated. For example, the doctor may select the location of build-up on an image and then the direction of the fluid spray may be automatically selected based on indicated image portion. In another example, the system may administer the fluid(s), detect any residue remaining (through human input or automatically), and then administer same or different fluid(s) again as needed.

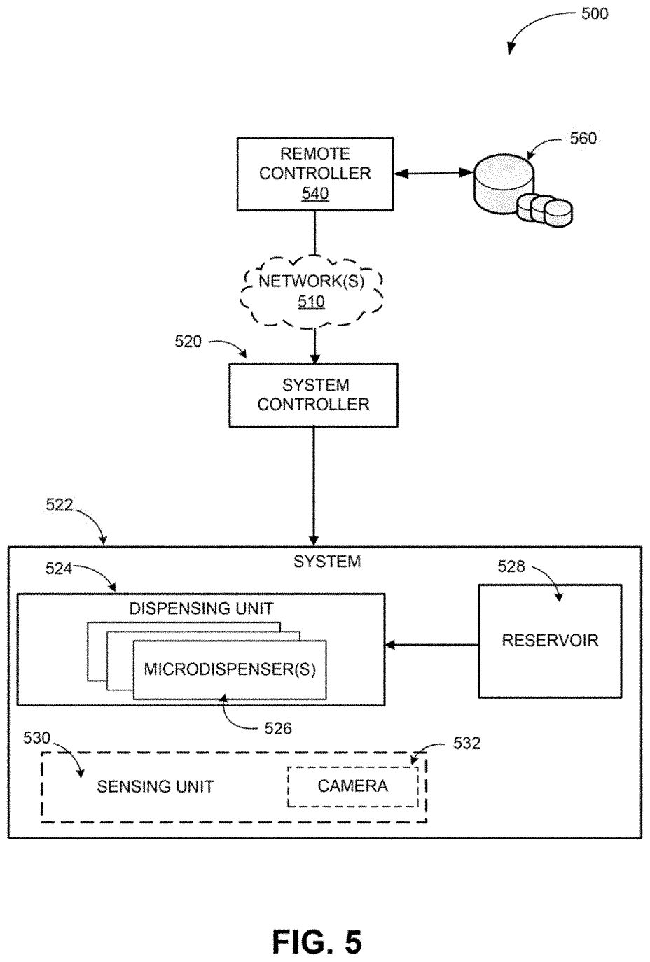

FIG. 5 is a block diagram illustrating major components in an example remotely controlled micro-dispenser based ear canal treatment system, arranged in accordance with at least some embodiments described herein.

A micro-dispenser based ear canal treatment system 522, as shown in diagram 500, may include a dispensing unit 524 with one or more micro-dispensers 526, a repository 528 with one or more reservoirs, and an optional sensing unit 530 with an optional camera 532 (or similar image detection device). A system controller 520 may receive input/instructions through manual input or from a remote controller 540 over one or more networks 510 to administer operations of the system 522 such as selection and adjustment of dispensation parameters, analysis and feedback of observations, etc. Data associated with the treatment and system parameters may be stored locally, as well as, in remote data stores 560.

In some examples, fluids may be dispensed into the ear canal in a controlled volume and pressure through one or more microdispensers to dissolve and remove ear wax in a gentle and safe manner without harming the eardrum. Through positioning of the microdispensers or nozzles of the microdispensers, the fluids may be aimed directly at specific areas within the ear canal with a high degree of precision. Different fluids may also be combined concurrently or sequentially to enhance effectiveness of the treatment. The microdispenser(s) and an optional imaging device such as a camera may be integrated with an otoscope or a cannula for manual manipulation as well as for aiming the fluid jet with visual assessment of treatment effectiveness.

The examples provided in FIGS. 1 through 5 are illustrated with specific systems, devices, applications, and scenarios. Embodiments are not limited to environments according to these examples. Treatment of ear canal may be implemented in environments employing fewer or additional systems, devices, applications, and scenarios. Furthermore, the example systems, devices, applications, and scenarios shown in FIGS. 1 through 5 may be implemented in a similar manner with other configurations using the principles described herein.

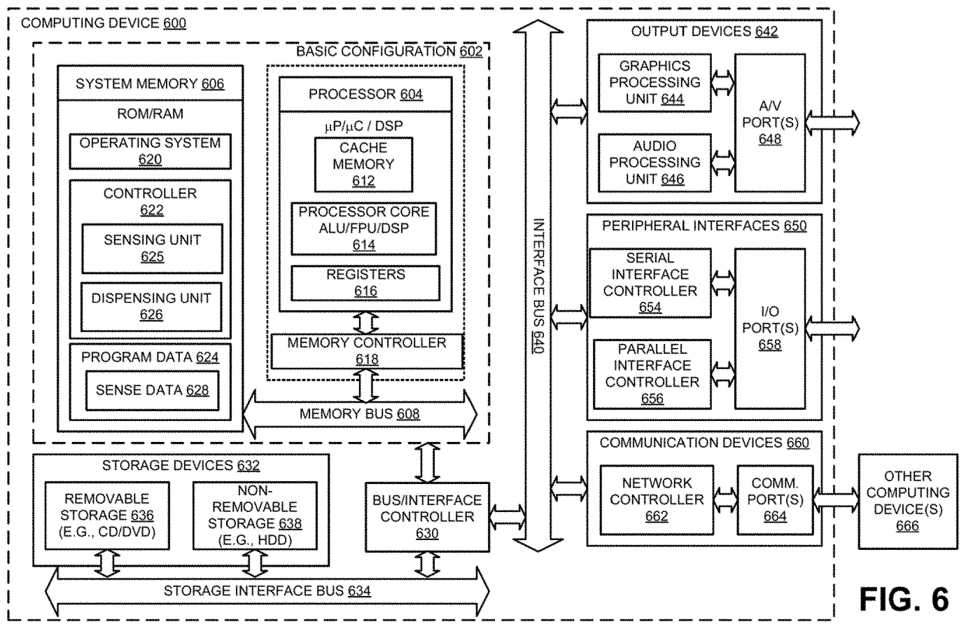

FIG. 6 illustrates a computing device, which may be used to control a micro-dispenser-based ear canal treatment system, arranged in accordance with at least some embodiments described herein.

In an example basic configuration 602, the computing device 600 may include one or more processors 604 and a system memory 606. A memory bus 608 may be used to communicate between the processor 604 and the system memory 606. The basic configuration 602 is illustrated in FIG. 6 by those components within the inner dashed line.

Depending on the desired configuration, the processor 604 may be of any type, including but not limited to a microprocessor (μP), a microcontroller (μC), a digital signal processor (DSP), or any combination thereof. The processor 604 may include one or more levels of caching, such as a cache memory 612, a processor core 614, and registers 616. The example processor core 614 may include an arithmetic logic unit (ALU), a floating point unit (FPU), a digital signal processing core (DSP Core), or any combination thereof. An example memory controller 618 may also be used with the processor 604, or in some implementations, the memory controller 618 may be an internal part of the processor 604.

Depending on the desired configuration, the system memory 606 may be of any type including but not limited to volatile memory (such as RAM), non-volatile memory (such as ROM, flash memory, etc.) or any combination thereof. The system memory 606 may include an operating system 620, a controller 622, and program data 624. The controller 622 may include a sensing unit 625 and a dispensing unit 626. The controller 622 may be configured to manage operations of the sensing unit 625 and the dispensing unit 626. For example, ear canal irrigating fluids may be dispensed in a controlled volume and pressure through one or more micro-dispensers of the dispensing unit 626 to dissolve and remove ear wax in a gentle and safe manner without harming the eardrum. The sensing unit 625 may be used to detect a location and amount of ear wax in order to adjust dispensing parameters. The program data 624 may include, among other data, sense data 628 or the like, as described herein.

The computing device 600 may have additional features or functionality, and additional interfaces to facilitate communications between the basic configuration 602 and any desired devices and interfaces. For example, a bus/interface controller 630 may be used to facilitate communications between the basic configuration 602 and one or more data storage devices 632 via a storage interface bus 634. The data storage devices 632 may be one or more removable storage devices 636, one or more non-removable storage devices 638, or a combination thereof. Examples of the removable storage and the non-removable storage devices include magnetic disk devices such as flexible disk drives and hard-disk drives (HDDs), optical disk drives such as compact disc (CD) drives or digital versatile disk (DVD) drives, solid state drives (SSDs), and tape drives to name a few. Example computer storage media may include volatile and nonvolatile, removable and non-removable media implemented in any method or technology for storage of information, such as computer readable instructions, data structures, program modules, or other data.

The system memory 606, the removable storage devices 636 and the non-removable storage devices 638 are examples of computer storage media. Computer storage media includes, but is not limited to, RAM, ROM, EEPROM, flash memory or other memory technology, CD-ROM, digital versatile disks (DVDs), solid state drives (SSDs), or other optical storage, magnetic cassettes, magnetic tape, magnetic disk storage or other magnetic storage devices, or any other medium which may be used to store the desired information and which may be accessed by the computing device 600. Any such computer storage media may be part of the computing device 600.

The computing device 600 may also include an interface bus 640 for facilitating communication from various interface devices (e.g., one or more output devices 642, one or more peripheral interfaces 650, and one or more communication devices 660) to the basic configuration 602 via the bus/interface controller 630. Some of the example output devices 642 include a graphics processing unit 644 and an audio processing unit 646, which may be configured to communicate to various external devices such as a display or speakers via one or more A/V ports 648. One or more example peripheral interfaces 650 may include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 660 includes a network controller 662, which may be arranged to facilitate communications with one or more other computing devices 666 over a network communication link via one or more communication ports 664. The one or more other computing devices 666 may include servers at a datacenter, customer equipment, and comparable devices.

The network communication link may be one example of a communication media. Communication media may be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

The computing device 600 may be implemented as a part of a general purpose or specialized server, mainframe, or similar computer that includes any of the above functions. The computing device 600 may also be implemented as a personal computer including both laptop computer and non-laptop computer configurations.

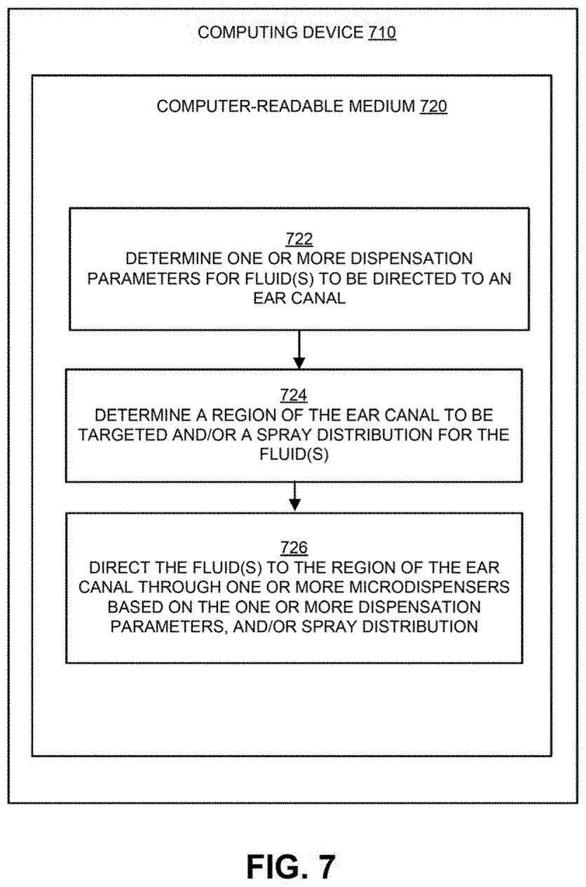

FIG. 7 is a flow diagram illustrating an example method to treat the ear canal through a micro-dispenser-based treatment system that may be performed by a computing device such as the computing device in FIG. 6, arranged in accordance with at least some embodiments described herein.

Example methods may include one or more operations, functions or actions as illustrated by one or more of blocks 722, 724, and/or 726, and may in some embodiments be performed or controlled by a computing device such as the computing device 710 in FIG. 7. The operations described in the blocks 722-726 may also be stored as computer-executable instructions in a computer-readable medium such as a computer-readable medium 720 of a computing device 710.

An example process to treat the ear canal through a micro-dispenser-based treatment system may begin with block 722, “DETERMINE ONE OR MORE DISPENSATION PARAMETERS FOR FLUID(S) TO BE DIRECTED TO AN EAR CANAL”, where dispensation parameters such as dispensed volume, pressure, direction, fluid temperature, dispensation pattern and timing, fluid composition, and/or nozzle selection may be determined through manual input, automatic computation based on observations, or a combination thereof.

Block 722 may be followed by block 724, “DETERMINE A REGION OF THE EAR CANAL TO BE TARGETED AND/OR A SPRAY DISTRIBUTION FOR THE FLUID”, where the direction of fluid spray (and optionally a spray pattern) may be determined based on a size of a build-up of an ear wax in the ear canal, a composition of the ear wax, a shape of the ear canal, a depth of the ear canal, and/or a thickness of the ear drum. The direction and pattern of the fluid spray may also be determined manually, automatically, or semi-automatically.

Block 724 may be followed by block 726, “DIRECT THE FLUID TO THE REGION OF THE EAR CANAL THROUGH ONE OR MORE MICRO-DISPENSERS BASED ON THE ONE OR MORE DISPENSATION PARAMETERS, AND/OR SPRAY DISTRIBUTION”, where the fluid(s) may be directed to the determined region using the selected/adjusted dispensation parameters. The desired direction, spray pattern, and other dispensation parameters may be applied through selection of micro-dispensers, nozzles, and other automatic or manual manipulations.

The operations included in the example process are for illustration purposes. Treatment of the ear canal through a micro-dispenser based system may be implemented by similar processes with fewer or additional steps, as well as in different order of operations using the principles described herein. The operations described herein may be executed by one or more processors operated on one or more computing devices, one or more processor cores, specialized processing devices, and/or general purpose processors, among other examples.

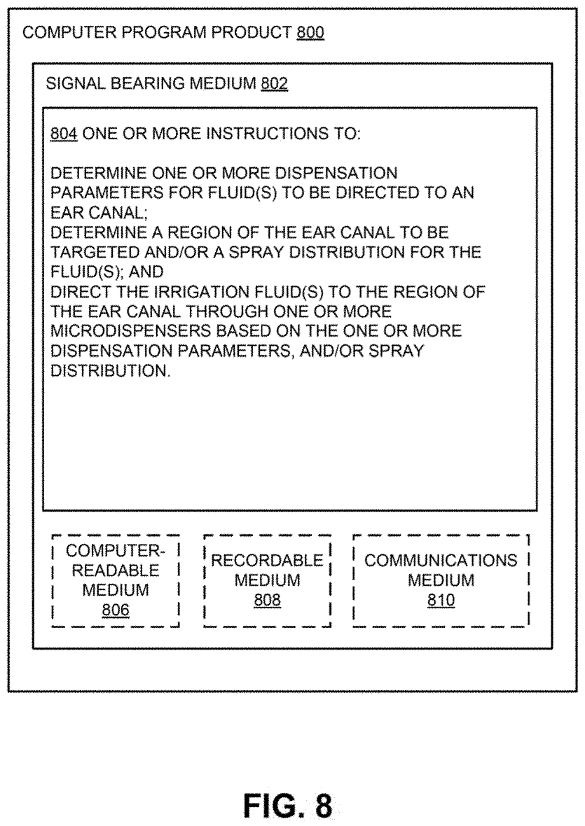

FIG. 8 illustrates a block diagram of an example computer program product, arranged in accordance with at least some embodiments described herein.

In some examples, as shown in FIG. 8, a computer program product 800 may include a signal bearing medium 802 that may also include one or more machine readable instructions 804 that, when executed by, for example, a processor may provide the functionality described herein. Thus, for example, referring to the processor 604 in FIG. 6, the controller 622 may undertake one or more of the tasks shown in FIG. 8 in response to the instructions 804 conveyed to the processor 604 by the signal bearing medium 802 to perform actions associated with determining one or more dispensation parameters for fluid(s) to be directed to an ear canal; determining a region of the ear canal to be targeted and/or a spray distribution for the fluid; and/or directing the fluid to the region of the ear canal through one or more micro-dispensers based on the one or more dispensation parameters, and/or spray distribution according to some embodiments described herein.

In some implementations, the signal bearing medium 802 depicted in FIG. 8 may encompass computer-readable medium 806, such as, but not limited to, a hard disk drive (HDD), a solid state drive (SSD), a compact disc (CD), a digital versatile disk (DVD), a digital tape, memory, etc. In some implementations, the signal bearing medium 802 may encompass recordable medium 808, such as, but not limited to, memory, read/write (R/W) CDs, R/W DVDs, etc. In some implementations, the signal bearing medium 802 may encompass communications medium 810, such as, but not limited to, a digital and/or an analog communication medium (e.g., a fiber optic cable, a waveguide, a wired communication link, a wireless communication link, etc.). Thus, for example, the computer program product 800 may be conveyed to one or more modules of the processor 804 by an RF signal bearing medium, where the signal bearing medium 802 is conveyed by the communications medium 810 (e.g., a wireless communications medium conforming with the IEEE 802.11 standard).

According to some examples, a method for ear canal treatment through a micro-dispenser-based system is described. The method may include determining one or more of a volume and a pressure for fluid to be directed to an ear canal; determining a region of the ear canal to be targeted; and directing the fluid to the region of the ear canal through one or more micro-dispensers based on the one or more of the volume and the pressure.

According to other examples, determining the region of the ear canal to be targeted may include determining the region through visual inspection or determining the region via inspection through a camera. Determining the one or more of the volume and the pressure for the fluid to be directed to the ear canal may include determining the one or more of the volume and the pressure for the fluid based on one or more of a size of a build-up of an ear wax in the ear canal, a composition of the ear wax, a shape of the ear canal, a depth of the ear canal, and a thickness of the ear drum.

According to further examples, the method may also include adjusting a shape of spray distribution for the fluid based on one or more of a location of a build-up of an ear wax in the ear canal, a size of the build-up of the ear wax in the ear canal, a composition of the ear wax, a shape of the ear canal, a depth of the ear canal, and a thickness of the ear drum. The method may further include adjusting the shape of spray distribution for the fluid by activating one or more nozzles associated with the one or more micro-dispensers. Directing the fluid to the region of the ear canal through the one or more micro-dispensers may include directing the fluid to the region of the ear canal through one or more nozzles positioned at a tip of a cannula. The method may also include determining an effectiveness of fluid dispensation via observation through a camera positioned on the cannula.

According to yet other examples, directing the fluid to the region of the ear canal through the one or more micro-dispensers may include directing the fluid to the region of the ear canal through one or more nozzles positioned at a tip of an otoscope. The method may further include determining an effectiveness of the fluid dispensation via observation through a camera positioned on the otoscope. Directing the fluid may include delivering a softening agent comprising one or more of plant-based oil, synthetic oil, urea hydrogen peroxide, glycerin, sodium bicarbonate solution in water, sodium bicarbonate and glycerin solution (B.P.C.), peanut oil turpentine and dichlorobenzene, triethanolamine polypeptides and oleate-condensate, warm water, saline solution, or water and vinegar solution. Directing the fluid further may also include delivering two or more softening agents or a softening agent and a rinsing solution through two or more distinct nozzles. The method may further include providing the fluid from a single reservoir to a plurality of micro-dispensers or providing the fluid from a single reservoir coupled to a single micro-dispenser.

According to other examples, a micro-dispenser based ear canal treatment system is described. The treatment system may include a delivery module comprising a supply sub-system and a dispensing sub-system, and a controller. The delivery module may direct a fluid to a selected region of an ear canal through one or more micro-dispensers. The controller may be configured to determine one or more of a volume and a pressure for the fluid to be directed to the ear canal; and determine the region of the ear canal to be targeted.

According to some examples, the controller may be configured to determine the region of the ear canal to be targeted based on manual manipulation of the treatment system. The controller may also be configured to determine the region of the ear canal to be targeted based on detection of a build-up of ear wax via inspection through a camera. The camera may be positioned on a cannula that includes one or more nozzles of one or more micro-dispensers positioned at a tip of the cannula. The camera may also be positioned on an otoscope that includes one or more nozzles of one or more micro-dispensers positioned at a tip of the otoscope. The controller may be configured to determine the one or more of the volume and the pressure for the fluid based on one or more of a size of a build-up of an ear wax in the ear canal, a composition of the ear wax, a shape of the ear canal, a depth of the ear canal, and a thickness of the ear drum.

According to other examples, the controller may be further configured to adjust a shape of spray distribution for the fluid based on one or more of a location of a build-up of an ear wax in the ear canal, a size of the build-up of the ear wax in the ear canal, a composition of the ear wax, a shape of the ear canal, a depth of the ear canal, and a thickness of the ear drum. The dispensing sub-system may be configured to deliver a softening agent comprising one or more of plant-based oil, synthetic oil, urea hydrogen peroxide, glycerin, sodium bicarbonate solution in water, sodium bicarbonate and glycerin solution (B.P.C.), peanut oil turpentine and dichlorobenzene, triethanolamine polypeptides and oleate-condensate, warm water, saline solution, or water and vinegar solution. The controller may be further configured to adjust one or more of the volume, the pressure, and a shape of spray distribution for the fluid based on a type of the softening agent.

According to yet other examples, the dispensing sub-system may be configured to deliver two or more softening agents or a softening agent and a rinsing solution through two or more distinct nozzles. The supply sub-system may include a single reservoir configured to provide the fluid to a dedicated micro-dispenser of the dispensing subs-system. The supply sub-system may also include a single reservoir configured to provide the fluid to a plurality of micro-dispensers of the dispensing subs-system. The system may further include a power module comprising one or more of a battery, a direct current power source, and an alternative current power source.

According to further examples, an otoscope capable of providing ear canal treatment is described. The otoscope may include one or more reservoirs configured to store one or more fluids and one or more micro-dispensers. The micro-dispensers may be configured to receive the one or more fluids from the one or more reservoirs; and direct the one or more fluids to a selected region of an ear canal at a predetermined volume and pressure through one or more nozzles, where the one or more nozzles are positioned at a tip of the otoscope.

According to other examples, the otoscope may further include a controller configured to determine the region of the ear canal to be targeted based on detection of a build-up of ear wax via inspection through a camera. The camera may be positioned at a tip of the otoscope. The controller may be configured to adjust the one or more of the volume and the pressure for the one or more fluids based on one or more of a size of a build-up of the ear wax in the ear canal, a composition of the ear wax, a shape of the ear canal, a depth of the ear canal, and a thickness of the ear drum. The controller may be further configured to adjust a shape of spray distribution for the one or more fluids based on one or more of a location of a build-up of the ear wax in the ear canal, a size of the build-up of the ear wax in the ear canal, a composition of the ear wax, a shape of the ear canal, a depth of the ear canal, and a thickness of the ear drum.

According to some examples, the one or more fluids may include a softening agent comprising one or more of plant-based oil, synthetic oil, urea hydrogen peroxide, glycerin, sodium bicarbonate solution in water, sodium bicarbonate and glycerin solution (B.P.C.), peanut oil turpentine and dichlorobenzene, triethanolamine polypeptides and oleate-condensate, warm water, saline solution, or water and vinegar solution. The controller may also be configured to adjust one or more of the volume, the pressure, and a shape of spray distribution for the one or more fluids based on a type of the softening agent. The one or more micro-dispensers may be configured to deliver two or more softening agents or a softening agent and a rinsing solution through two or more distinct nozzles. The otoscope may also include a power module comprising one or more of a battery, a direct current power source, and an alternative current power source.

According to some examples, a cannula capable of providing ear canal treatment is described. The cannula may include one or more micro-dispensers coupled to one or more reservoirs configured to store one or more fluids. The one or more micro-dispensers may be configured to receive the one or more fluids from the one or more reservoirs; and direct the one or more fluids to a selected region of an ear canal at a predetermined volume and pressure through one or more nozzles, where the one or more nozzles are positioned at a tip of the cannula.

According to further examples, the one or more reservoirs may be external to the cannula or integrated with the cannula. The cannula may further include a controller configured to determine the region of the ear canal to be targeted based on detection of a build-up of ear wax via inspection through a camera. The camera may be positioned at a tip of the cannula. The controller may be configured to adjust the one or more of the volume and the pressure for the one or more fluids based on one or more of a size of a build-up of the ear wax in the ear canal, a composition of the ear wax, a shape of the ear canal, a depth of the ear canal, and a thickness of the ear drum. The controller may be further configured to adjust a shape of spray distribution for the one or more fluids based on one or more of a location of a build-up of the ear wax in the ear canal, a size of the build-up of the ear wax in the ear canal, a composition of the ear wax, a shape of the ear canal, a depth of the ear canal, and a thickness of the ear drum.

According to some examples, the one or more fluids may include a softening agent comprising one or more of plant-based oil, synthetic oil, urea hydrogen peroxide, glycerin, sodium bicarbonate solution in water, sodium bicarbonate and glycerin solution (B.P.C.), peanut oil turpentine and dichlorobenzene, triethanolamine polypeptides and oleate-condensate, warm water, saline solution, or water and vinegar solution. The controller may also be configured to adjust one or more of the volume, the pressure, and a shape of spray distribution for the one or more fluids based on a type of the softening agent. The one or more micro-dispensers may be configured to deliver two or more softening agents or a softening agent and a rinsing solution through two or more distinct nozzles. The cannula may also include a power module comprising one or more of a battery, a direct current power source, and an alternative current power source.

There is little distinction left between hardware and software implementations of aspects of systems; the use of hardware or software is generally (but not always, in that in certain contexts the choice between hardware and software may become significant) a design choice representing cost vs. efficiency tradeoffs. There are various vehicles by which processes and/or systems and/or other technologies described herein may be affected (e.g., hardware, software, and/or firmware), and that the preferred vehicle will vary with the context in which the processes and/or systems and/or other technologies are deployed. For example, if an implementer determines that speed and accuracy are paramount, the implementer may opt for mainly hardware and/or firmware vehicle; if flexibility is paramount, the implementer may opt for mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples may be implemented, individually and/or collectively, by a wide range of hardware, software, firmware, or virtually any combination thereof. In one embodiment, several portions of the subject matter described herein may be implemented via application specific integrated circuits (ASICs), field programmable gate arrays (FPGAs), digital signal processors (DSPs), or other integrated formats. However, those skilled in the art will recognize that some aspects of the embodiments disclosed herein, in whole or in part, may be equivalently implemented in integrated circuits, as one or more computer programs executing on one or more computers (e.g., as one or more programs executing on one or more computer systems), as one or more programs executing on one or more processors (e.g., as one or more programs executing on one or more microprocessors), as firmware, or as virtually any combination thereof, and that designing the circuitry and/or writing the code for the software and/or firmware would be well within the skill of one of skill in the art in light of this disclosure.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is also to be understood that the terminology used herein is for the purpose of describing particular embodiments only, and is not intended to be limiting.

In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive (HDD), a compact disc (CD), a digital versatile disk (DVD), a digital tape, a computer memory, a solid state drive (SSD), etc.; and a transmission type medium such as a digital and/or an analog communication medium (e.g., a fiber optic cable, a waveguide, a wired communication link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein may be integrated into a data processing system via a reasonable amount of experimentation. Those having skill in the art will recognize that a data processing system may include one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors.

A data processing system may be implemented utilizing any suitable commercially available components, such as those found in data computing/communication and/or network computing/communication systems. The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermediate components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically connectable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general, such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A micro-dispenser based ear canal treatment system, comprising:
a delivery module comprising a supply sub-system and a dispensing sub-system, wherein the delivery module is configured to:
direct a fluid to a selected region of an ear canal through one or more micro-dispensers; and
a controller coupled to the delivery module, the controller configured to:
determine one or more dispensation parameters for the fluid to be directed to the ear canal; and
determine the region of the ear canal to be targeted.

2. The treatment system of claim 1, further comprising a camera coupled to the controller, wherein the controller is configured to determine the region of the ear canal to be targeted based on one or more of: manual manipulation of the treatment system or detection of a build-up of ear wax via inspection through the camera.

3. (canceled)

4. The treatment system of claim 1, wherein the controller is configured to determine the region of the ear canal to be targeted based on detection of a build-up of ear wax via inspection through a camera, and wherein the camera is part of:
a cannula that includes one or more nozzles of one or more micro-dispensers positioned at a tip of the cannula; or
an otoscope that includes one or more nozzles of one or more micro-dispensers positioned at a tip of the otoscope.

5. (canceled)

6. The treatment system of claim 1, wherein the dispensation parameters comprise one or more of: a dispensed volume, a pressure, a direction, a fluid temperature, a dispensation pattern, a fluid composition, or a nozzle selection.

7. The treatment system of claim 6, wherein the controller is configured to determine the one or more dispensation parameters based on one or more of: a size of a build-up of an ear wax in the ear canal, a composition of the ear wax, a shape of the ear canal, a depth of the ear canal, and a thickness of the ear drum.

8. The treatment system of claim 1, wherein the controller is further configured to:
adjust a shape of spray distribution for the fluid based on one or more of: a location of a build-up of an ear wax in the ear canal, a size of the build-up of the ear wax in the ear canal, a composition of the ear wax, a shape of the ear canal, a depth of the ear canal, and a thickness of the ear drum.

9. The treatment system of claim 1, wherein the dispensing sub-system is configured to:
deliver a softening agent comprising one or more of: plant-based oil, synthetic oil, urea hydrogen peroxide, glycerin, sodium bicarbonate solution in water, sodium bicarbonate and glycerin solution (B.P.C.), peanut oil turpentine and dichlorobenzene, triethanolamine polypeptides and oleate-condensate, warm water, saline solution, or water and vinegar solutions; and
adjust one or more of: the volume, the pressure, and a shape of spray distribution for the fluid based on a type of the softening agent.

10. (canceled)

11. The treatment system of claim 1, wherein the dispensing sub-system is configured to:
deliver two or more softening agents or a softening agent and a rinsing solution through two or more distinct nozzles.

12. The treatment system of claim 1, wherein the supply sub-system includes a single reservoir configured to:
provide the fluid to a dedicated micro-dispenser of the dispensing subs-system; or
provide the fluid to a plurality of micro-dispensers of the dispensing subs-system.

13. (canceled)

14. The treatment system of claim 1, further comprising:
a power module comprising one or more of: a battery, a direct current power source, and an alternative current power source.

15. A method for ear canal treatment through a micro-dispenser-based system, the method comprising:
determining one or more dispensation parameters for a fluid to be directed to an ear canal;
determining a region of the ear canal to be targeted; and
directing the fluid to the region of the ear canal through one or more micro-dispensers based on the one or more dispensation parameters.

16. The method of claim 15, wherein determining the region of the ear canal to be targeted comprises:
determining the region through visual inspection; or
determining the region via inspection through a camera.

17. (canceled)

18. The method of claim 15, wherein determining the one or more dispensation parameters for the fluid to be directed to the ear canal comprises:
determining one or more of a dispensed volume, a pressure, a direction, a fluid temperature, a dispensation pattern, a fluid composition, or a nozzle selection.

19. The method of claim 18, wherein determining the one or more dispensation parameters for the fluid to be directed to the ear canal comprises:
determining the one or more dispensation parameters based on one or more of: a size of a build-up of an ear wax in the ear canal, a composition of the ear wax, a shape of the ear canal, a depth of the ear canal, and a thickness of the ear drum.

20. The method of claim 15, further comprising:
adjusting a shape of spray distribution for the fluid based on one or more of: a location of a build-up of an ear wax in the ear canal, a size of the build-up of the ear wax in the ear canal, a composition of the ear wax, a shape of the ear canal, a depth of the ear canal, and a thickness of the ear drum; and
adjusting the shape of spray distribution for the fluid by activating one or more nozzles associated with the one or more micro-dispensers.

21. (canceled)

22. The method of claim 15, wherein directing the fluid to the region of the ear canal through the one or more micro-dispensers comprises:
directing the fluid to the region of the ear canal through one or more nozzles positioned at a tip of a cannula or a tip of an otoscope.

23. The method of claim 22, further comprising:
determining an effectiveness of fluid dispensation via observation through a camera positioned on the cannula or on the otoscope.

24. (canceled)

25. (canceled)

26. The method of claim 15, wherein directing the fluid comprises:
delivering a softening agent comprising one or more of plant-based oil, synthetic oil, urea hydrogen peroxide, glycerin, sodium bicarbonate solution in water, sodium bicarbonate and glycerin solution (B.P.C.), peanut oil turpentine and dichlorobenzene, triethanolamine polypeptides and oleate-condensate, warm water, saline solution, or water and vinegar solution.

27. The method of claim 15, wherein directing the fluid further comprises:
delivering two or more softening agents or a softening agent and a rinsing solution through two or more distinct nozzles.

28. The method of claim 15, further comprising:
providing the fluid from a single reservoir to a plurality of micro-dispensers or from a single reservoir coupled to a single micro-dispenser.

29. (canceled)

30. An otoscope capable of providing ear canal treatment, the otoscope comprising:
one or more reservoirs configured to store one or more fluids;
one or more micro-dispensers, configured to:
receive the one or more fluids from the one or more reservoirs; and
direct the one or more fluids to a selected region of an ear canal based on one or more dispensation parameters through one or more nozzles, wherein the one or more nozzles are positioned at a tip of the otoscope.

31. The otoscope of claim 30, further comprising:
a camera; and
a controller coupled to the camera, the controller configured to:
determine the region of the ear canal to be targeted based on detection of a build-up of ear wax via inspection through the camera, wherein the camera is positioned at a tip of the otoscope.

32. (canceled)

33. The otoscope of claim 31, wherein the one or more dispensation parameters comprise a dispensed volume, a pressure, a direction, a fluid temperature, a dispensation pattern, a fluid composition, or a nozzle selection, and the controller is further configured to:
adjust the one or more dispensation parameters for the one or more fluids based on one or more of: a size of a build-up of the ear wax in the ear canal, a composition of the ear wax, a shape of the ear canal, a depth of the ear canal, and a thickness of the ear drum; or
adjust a shape of spray distribution for the one or more fluids based on one or more of: a location of a build-up of the ear wax in the ear canal, a size of the build-up of the ear wax in the ear canal, a composition of the ear wax, a shape of the ear canal, a depth of the ear canal, and a thickness of the ear drum.

34. (canceled)

35. (canceled)

36. The otoscope of claim 31, wherein the one or more fluids include a softening agent comprising one or more of: plant-based oil, synthetic oil, urea hydrogen peroxide, glycerin, sodium bicarbonate solution in water, sodium bicarbonate and glycerin solution (B.P.C.), peanut oil turpentine and dichlorobenzene, triethanolamine polypeptides and oleate-condensate, warm water, saline solution, or water and vinegar solution, and the controller is further configured to:
adjust one or more of: the volume, the pressure, and a shape of spray distribution for the one or more fluids based on a type of the softening agent.

37. (canceled)

38. The otoscope of claim 30, wherein the one or more micro-dispensers are configured to:
deliver two or more softening agents or a softening agent and a rinsing solution through two or more distinct nozzles.

39. The otoscope of claim 30, further comprising:
a power module comprising one or more of: a battery, a direct current power source, and an alternative current power source.

40. A cannula capable of providing ear canal treatment, the cannula comprising:
one or more micro-dispensers coupled to one or more reservoirs configured to store one or more fluids, the one or more micro-dispensers configured to:
receive the one or more fluids from the one or more reservoirs; and
direct the one or more fluids to a selected region of an ear canal based on one or more dispensation parameters through one or more nozzles, wherein the one or more nozzles are positioned at a tip of the cannula and wherein the one or more dispensation parameters comprise a dispensed volume, a pressure, a direction, a fluid temperature, a dispensation pattern, a fluid composition, or a nozzle selection.

41. The cannula of claim 40, wherein the one or more reservoirs are external to the cannula.

42. The cannula of claim 40, wherein the one or more reservoirs are integrated with the cannula.

43. (canceled)

44. The cannula of claim 40, further comprising:
a camera; and
a controller coupled to the camera, the controller configured to:
determine the region of the ear canal to be targeted based on detection of a build-up of ear wax via inspection through the camera, wherein the camera is positioned at a tip of the cannula.

45. (canceled)

46. The cannula of claim 44, wherein the controller is further configured to:
adjust the one or more dispensation parameters for the one or more fluids based on one or more of: a size of a build-up of the ear wax in the ear canal, a composition of the ear wax, a shape of the ear canal, a depth of the ear canal, and a thickness of the ear drum; or
adjust a shape of spray distribution for the one or more fluids based on one or more of: a location of a build-up of the ear wax in the ear canal, a size of the build-up of the ear wax in the ear canal, a composition of the ear wax, a shape of the ear canal, a depth of the ear canal, and a thickness of the ear drum.

47. (canceled)

48. The cannula of claim 44, wherein the one or more fluids include a softening agent comprising one or more of plant-based oil, synthetic oil, urea hydrogen peroxide, glycerin, sodium bicarbonate solution in water, sodium bicarbonate and glycerin solution (B.P.C.), peanut oil turpentine and dichlorobenzene, triethanolamine polypeptides and oleate-condensate, warm water, saline solution, or water and vinegar solution, and the controller is further configured to:
adjust one or more of: the volume, the pressure, and a shape of spray distribution for the one or more fluids based on a type of the softening agent.

49. (canceled)

50. The cannula of claim 40, wherein the one or more micro-dispensers are configured to:
deliver two or more softening agents or a softening agent and a rinsing solution through two or more distinct nozzles.

51. The cannula of claim 40, further comprising:
a power module comprising one or more of: a battery, a direct current power source, and an alternative current power source.
